(19) United States
(12) Patent Application Publication
(10) Pub. No.: US 2023/0349001 A1
(43) Pub. Date: Nov. 2, 2023

(54) BIOMOLECULAR CHARACTERISTIC IDENTIFICATION METHOD AND SYSTEM THEREOF FOR TAIWAN TILAPIA STRAINS

(71) Applicant: National Taiwan Ocean University, Keelung (TW)

(72) Inventors: CHANG-WEN HUANG, Keelung (TW); TE-HUA HSU, Keelung (TW); HONG-YI GONG, Keelung (TW)

(21) Appl. No.: 17/848,170

(22) Filed: Jun. 23, 2022

(30) Foreign Application Priority Data
May 2, 2022 (TW) 111116588

(51) Int. Cl. C12Q 1/6888

(52) U.S. Cl. CPC C12Q 1/6888; C12Q 1/686; C12Q 2600/124; C12Q 2600/158

(57) ABSTRACT

A biomolecular characteristic identification method includes: breeding a plurality of Taiwan Tilapias in a predetermined environment to obtain a plurality of baited Taiwan Tilapias; seeking at least one nucleotide mark in the baited Taiwan Tilapias to obtain at least one feature sequence marker therefrom; producing at least one design of primer pair according to the feature sequence marker for identification; and identifying an unknown DNA sample of the baited Taiwan Tilapias with the at least one primer pair in a biomolecular tracing procedure to obtain an identification result.

### SEQUENCE LISTING

This sequence listing is created on Jun. 23, 2022 with the file name “P220014TWTOU_SF22_0004UP_ST25” and file size 14kb; the entire contents of which are hereby incorporated by reference.

### BACKGROUND OF THE INVENTION

### 1. Field of the Invention

The present invention relates to a biomolecular characteristic identification method and system thereof for Taiwan Tilapia strains. Particularly, the present invention relates to the biomolecular characteristic identification method and system thereof for Taiwan Tilapia strains with primer pairs. More particularly, the present invention relates to the biomolecular characteristic (e.g., cold hardiness, disease resistance and salinity tolerance) identification method and system (or information system) thereof for Taiwan Tilapia strains.

### 2. Description of the Related Art

By way of example, Chinese Patent Application Publication No. CN-107871060, entitled “Canine strain identification method based on DNA marker,” discloses a method for accurately identifying canine strain of a pet dog DNA SNP (Single Nucleotide Polymorphism) marker.

The canine strain identification method includes the steps: (1) obtaining pet dog DNA SNP marker data of unknown canine strain; (2) carrying out hierarchical clustering on the SNP marker data obtained in step (1), wherein migratory data or parameters are added to the hierarchical clustering.

The canine strain identification method further includes the steps: (3) carrying out population mixture analysis on the SNP marker data obtained in step (1), wherein the amount of hybrid strains is restricted in the population mixture analysis.

The canine strain identification method further includes the steps: (4) dividing a genome into small areas, carrying out dog category classification on each area by a random forest algorithm, utilizing a hidden Markov chain to smooth the strain classification of the small gene area, utilizing maximum likelihood estimation to estimate a family pedigree, obtaining the strain hybrid ingredients of dog parents, and accomplishing pedigree deduction.

Further, another Chinese Patent Publication No. CN-07254551, entitled “Method of identifying and breeding for novel inbred strains of PERV-pol gene-deficient Wuzhishan miniature pigs,” discloses a method of identifying for inbred strains of PERV-pol (Porcine Endogenous Retroviruses-pol) gene-deficient Wuzhishan miniature pigs and a method of breeding for inbred strains of PERV-pol gene-deficient Wuzhishan miniature pigs.

The method of identifying and breeding for novel inbred strains of PERV-pol gene-deficient Wuzhishan miniature pigs, with using a reagent kit having a material “A”, includes: (a) identifying inbred strains of PERV-pol gene-deficient Wuzhishan miniature pigs; and (b) breeding inbred strains of PERV-pol gene-deficient Wuzhishan miniature pigs.

The material “A”, provided in the reagent kit, is a material capable of detecting whether an early termination substance appearing a PERV-pol gene in whole genome of an inbred strain of PERV-pol gene-deficient Wuzhishan miniature pigs.

Further, another Chinese Patent Publication No. CN-109022592, entitled “SNP markers for identification of rats from four commonly-used strains and application thereof,” discloses a SNP marker for identification of rats from four commonly-used strains and application thereof, with the SNP markers including 13 SNP sites.

The 13 SNP sites of the SNP markers are SNP1 to SNP13 respectively that indicate that the 13 SNP sites are located at position 201 of nucleotide sequences shown in SEQ ID No. 1 to SEQ ID No. 13. The SNP markers can be used for rapid identification of rats from four commonly-used strains (i.e., Wistar, GK, BN and SD).

Further, another PCT (Patent Cooperation Treaty) Patent Application Publication No. WO-2011/108062, entitled “Method for labeling and identifying fishery fish and shellfish by mitochondrial DNA variable region base sequence,” discloses a method for labeling and identifying fishery fish and shellfish by mitochondrial DNA variable region base sequence.

The method for labeling and identification of fishery fish and shellfish is a simple and accurate method for labeling and identification of fish and shellfish, particularly fishery fish and shellfish derived from a specific production area, or superior fishery eggs and fry having one or a plurality of superior characterizing features, or a specific type of fishery fish and shellfish.

The method for labeling and identification of fishery fish and shellfish is a method for labeling and determining or tracking the profile of a single fishery fish or shellfish, an individual school of fishery fish or shellfish, a portion thereof, or the processed product thereof, by using the D loop base sequence of mitochondrial DNA. The method is also provided to label fishery fish and shellfish, particularly fertilized eggs, juvenile fish, or fingerlings.

However, there is a need of improving the conventional method for labeling and identification of fishery fish and shellfish performed as an identification of unique nucleotide marker for aquatic creatures and products. The above-mentioned patents and patent application publications are incorporated herein by reference for purposes including, but not limited to, indicating the background of the present invention and illustrating the situation of the art.

### SUMMARY OF THE INVENTION

The primary objective of this invention is to provide a biomolecular characteristic identification method and system thereof for Taiwan Tilapia strains. A plurality of Taiwan Tilapias are bred and trained in a predetermined (i.e., conditioned) environment to obtain a plurality of baited Taiwan Tilapias, with seeking at least one nucleotide mark in the baited Taiwan Tilapias to obtain at least one feature sequence marker therefrom, with producing at least one design of primer pair according to the feature sequence marker for identification, with identifying a DNA sample of the baited Taiwan Tilapias with the at least one primer pair in a biomolecular tracing procedure to obtain an identification result. Advantageously, the biomolecular characteristic identification method and system of the present invention is successful in tracing original aquatic creatures and products thereof.

The biomolecular characteristic identification system in accordance with an aspect of the present invention includes:
a predetermined environment conditioned and provided to breed and train a plurality of Taiwan Tilapias to obtain a plurality of baited Taiwan Tilapias;
at least one feature sequence marker obtained from seeking at least one nucleotide mark in the baited Taiwan Tilapias;
at least one designed primer pair corresponding to the feature sequence marker for identification and produced according to the feature sequence marker; and
an identification result obtained from identifying an unknown DNA sample of the baited Taiwan Tilapias with the at least one primer pair in a biomolecular tracing procedure;
wherein the identification result shows a correct source of original baited Taiwan Tilapias or an incorrect source of original baited Taiwan Tilapias.

The biomolecular characteristic identification method in accordance with an aspect of the present invention includes:
breeding and training a plurality of Taiwan Tilapias in a predetermined environment to obtain a plurality of baited Taiwan Tilapias;
seeking at least one nucleotide mark in the baited Taiwan Tilapias to obtain at least one feature sequence marker therefrom;
producing at least one design of primer pair according to the feature sequence marker for identification; and
identifying an unknown DNA sample of the baited Taiwan Tilapias with the at least one primer pair in a biomolecular tracing procedure to obtain an identification result.

In a separate aspect of the present invention, the feature sequence marker is selected from a cold hardiness marker, a disease resistance marker, a salinity tolerance marker or combinations thereof.

In a further separate aspect of the present invention, the cold hardiness marker is selected from a (ATT)ₙ-sequence-contained marker, a (G)ₙ-sequence-contained marker, a (GAG)ₙ-sequence-contained marker, a (GT)ₙ-sequence-contained marker or a (CA)ₙ-sequence-contained marker.

In yet a further separate aspect of the present invention, the salinity tolerance marker is selected from a (CA)ₙ-sequence-contained marker, a (AT)₈-sequence-contained marker, a (TAT)₈-sequence-contained marker, a (TTTG)₅-sequence-contained marker or a (AT)₁₂-sequence-contained marker.

In yet a further separate aspect of the present invention, the disease resistance marker is selected from a (CAGG)₇-sequence-contained marker, a (CTAC)₇-sequence-contained marker, a (TTGA)₂₀-sequence-contained marker, a (TTGT)₁₀-sequence-contained marker or a (GAAAA)₆-sequence-contained marker.

The exogenous biomolecular tracing method in accordance with another aspect of the present invention includes:
extracting a first nucleotide marker from a first marker source organism;
combining a first basic material with the first nucleotide marker to form a first nucleotide marker-contained material;
extracting a second nucleotide marker from a second marker source organism;
combining a second basic material with the second nucleotide marker to form a second nucleotide marker-contained material;
in a first coding stage, exogenously combining the first nucleotide marker-contained material with an aquatic creature or an aquatic product to form a first exogenously-marked aquatic creature or a first exogenously-marked aquatic product;
in a second coding stage, exogenously combining the second nucleotide marker-contained material with the first exogenously-marked aquatic creature or the first exogenously-marked aquatic product to form a second exogenously-marked aquatic creature or a second exogenously-marked aquatic product; and
identifying a DNA sample received from the second exogenously-marked aquatic creature or the second exogenously-marked aquatic product with at least one primer pair in an exogenous biomolecular tracing procedure to obtain an exogenously-marked identification result.

In a separate aspect of the present invention, the first or second nucleotide marker is selected from a DNA extract, a DNA extract liquid, a DNA extract powder, a DNA extract-related material or combinations thereof.

In a further separate aspect of the present invention, exogenously combining the first or second nucleotide marker-contained material with the aquatic creature or the aquatic product includes a feeding process, a dipping process, a painting process, a spraying process or combinations thereof.

In yet a further separate aspect of the present invention, the at least one primer pair is applied to execute a test of polymerase chain reaction (PCR) or loop-mediated isothermal amplification (LAMP).

In yet a further separate aspect of the present invention, the exogenously-marked identification result includes a combination set of codes (positive identified as “+” or code “1” and negative identified as “-” or code “0”) or code forms for tracing the second exogenously-marked aquatic creature or the second exogenously-marked aquatic product.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic block diagram of a biomolecular characteristic identification system for Taiwan Tilapia strains in accordance with a preferred embodiment of the present invention.

FIG. 2 is a schematic block diagram of a series of feature sequence markers of Taiwan Tilapia applied in the biomolecular characteristic identification method and system thereof for Taiwan Tilapia strains in accordance with a preferred embodiment of the present invention.

FIG. 3 is a flow chart of a biomolecular characteristic identification method for Taiwan Tilapia strains in accordance with a preferred embodiment of the present invention.

FIG. 4 is a schematic block diagram of an exogenous biomolecular tracing system applied in the biomolecular characteristic identification method and system thereof for Taiwan Tilapia strains in accordance with a preferred embodiment of the present invention.

FIG. 5 is a flow chart of an exogenous biomolecular tracing system applied in the biomolecular characteristic identification method and system thereof for Taiwan Tilapia strains in accordance with a preferred embodiment of the present invention.

### DETAILED DESCRIPTION OF THE INVENTION

It is noted that a biomolecular characteristic identification method (or usage) and system (e.g., information system) thereof for Taiwan Tilapia strains in accordance with the preferred embodiment of the present invention can be applicable to various aquatic creature baiting systems, various aquatic fry or creature breeding systems, various aquatic creature display systems, various algae (or microalgae) cultivation systems or various algae display systems, which are not limitative of the present invention.

For instance, a biomolecular characteristic identification method (or usage) and system (e.g., information system) thereof for Taiwan Tilapia strains in accordance with the preferred embodiment of the present invention can be suitable for a Taiwan Tilapia strain (National Taiwan Ocean University), including Taiwan Tilapia (A), Taiwan Tilapia (Y), Taiwan Tilapia (B), Taiwan Tilapia (T) or other related Taiwan Tilapia strains, which are not limitative of the present invention.

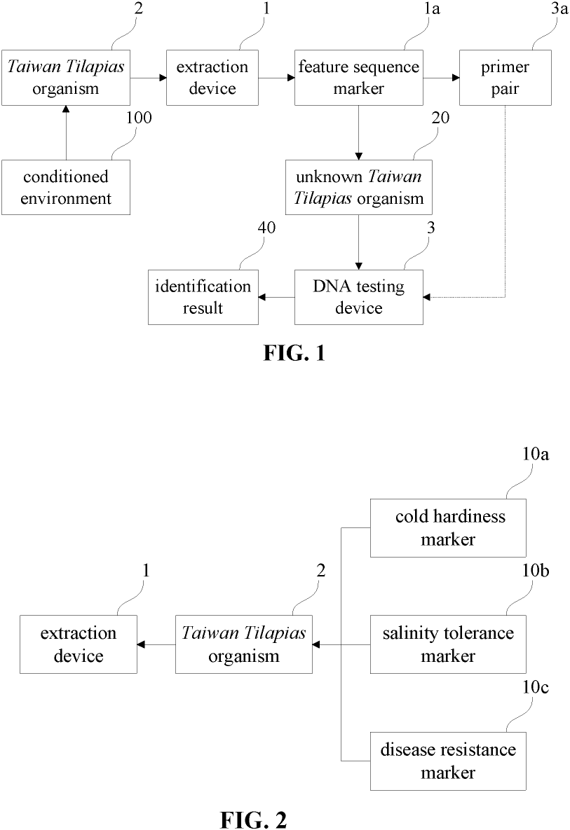

FIG. 1 shows a schematic block diagram of a biomolecular characteristic identification system for Taiwan Tilapia strains in accordance with a preferred embodiment of the present invention. Referring now to FIG. 1, the biomolecular characteristic identification system in accordance with the preferred embodiment of the present invention includes a predetermined environment 100, at least one feature sequence marker 1a, a plurality of Taiwan Tilapia organism 2 and at least one primer pair 3a.

With continued reference to FIG. 1, by way of example, the predetermined environment 100 can be selected from a cage culture site, an aquaculture outdoor site, an aquaculture tank, an aquaculture container or the likes, and controlled by various conditions, including temperature parameters, salinity parameters, pathogenic bacteria parameters or other environment conditions for training Taiwan Tilapia.

Still referring to FIG. 1, by way of example, the Taiwan Tilapia organism 2 can be selected from predetermined strains of Taiwan Tilapia (A), Taiwan Tilapia (Y), Taiwan Tilapia (B), Taiwan Tilapia (T) or other similar strains.

With continued reference to FIG. 1, by way of example, in another embodiment, the biomolecular characteristic identification system further includes at least one extraction device 1, at least one DNA testing device 3 and related peripheral devices, e.g. feeding machine, painting machine, spraying machine or other machines.

FIG. 2 shows a schematic block diagram of a series of feature sequence markers of Taiwan Tilapia applied in the biomolecular characteristic identification method and system thereof for Taiwan Tilapia strains in accordance with a preferred embodiment of the present invention. Turning now to FIGS. 1 and 2, by way of example, the Taiwan Tilapia organism 2 has at least one feature sequence marker, including a cold hardiness marker, a disease resistance marker, a salinity tolerance marker or combinations thereof.

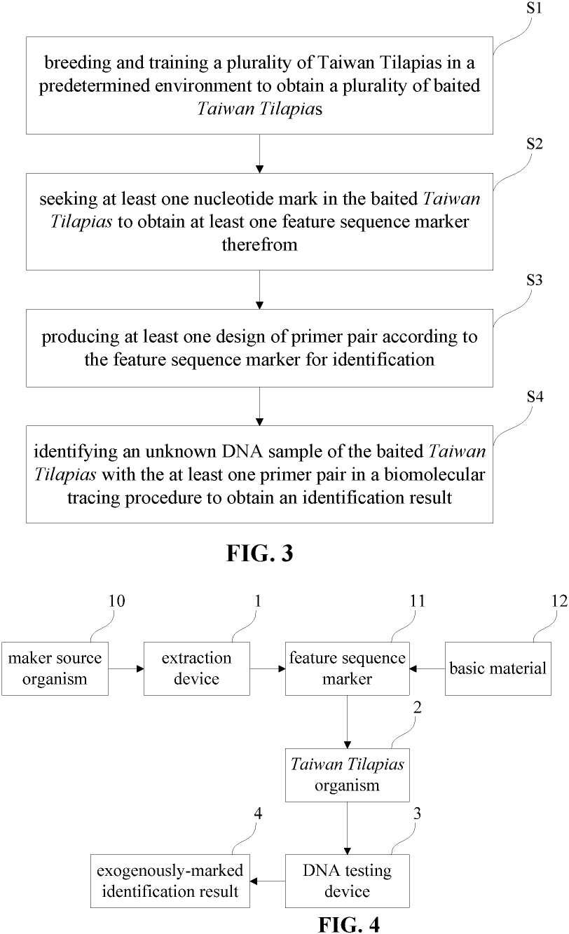

FIG. 3 shows a flow chart of a biomolecular characteristic identification method for Taiwan Tilapia strains in accordance with a preferred embodiment of the present invention. Turning now to FIGS. 1, 2 and 3, by way of example, the biomolecular characteristic identification method in accordance with the preferred embodiment of the present invention includes the step S1: automatically, semi-automatically or manually breeding and training a plurality of Taiwan Tilapias (e.g., Taiwan Tilapias organism 2) in the predetermined environment 100 with various conditions to obtain a plurality of baited Taiwan Tilapias.

Still referring to FIGS. 1, 2 and 3, by way of example, the biomolecular characteristic identification method in accordance with the preferred embodiment of the present invention includes the step S2: automatically, semi-automatically or manually seeking at least one nucleotide mark in the baited Taiwan Tilapias to obtain the at least one feature sequence marker 1a (e.g., cold hardiness marker, disease resistance marker, salinity tolerance marker) therefrom.

Still referring to FIGS. 1, 2 and 3, by way of example, the biomolecular characteristic identification method in accordance with the preferred embodiment of the present invention includes the step S3: automatically, semi-automatically or manually producing at least one design of primer pair (i.e., artificial sequence) 3a according to the feature sequence marker 1a for identification with it.

Still referring to FIGS. 1, 2 and 3, by way of example, the biomolecular characteristic identification method in accordance with the preferred embodiment of the present invention includes the step S4: automatically, semi-automatically or manually identifying an unknown DNA sample of the baited Taiwan Tilapias (i.e., unknown Taiwan Tilapias organism 20) with the at least one primer pair 3a in a biomolecular tracing procedure to obtain an identification result 40.

With continued reference to FIGS. 1, 2 and 3, by way of example, a DNA sample of unknown Taiwan Tilapias organism 20 is extracted in the extraction device 1 and can be tested by the DNA testing device 3 with various suitable manners, such as: electrophoresis, capillary electrophoresis, PCR-RFLP (polymerase chain reaction-restriction fragment length polymorphism) or other suitable manners.

Still referring to FIGS. 1, 2 and 3, by way of example, the nucleotide mark can be selected from various cold hardiness markers 10a (e.g., Seq. 1, Seq. 2, Seq. 5, Seq. 6, Seq. 9, Seq. 10, Seq. 13, Seq. 14, Seq. 17, Seq. 18), various salinity tolerance markers 10b (e.g., Seq. 21, Seq. 24, Seq. 27, Seq. 30, Seq. 33), various disease resistance markers 10c (e.g., Seq. 36, Seq. 39, Seq. 42, Seq. 45, Seq. 48).

By way of example, the cold hardiness marker can be selected from a (ATT)ₙ-sequence-contained marker as follows:

Seq. 1:GACTACATCCCAGCAACAGTGACACATTCCACCGCGAGCCCAC
CCTCCACCTTCCCAACACTACCATTATTATTATTATTATTATTATTATTA TTATTATTATTATTATTTGTATTTTTTTACTTGGAGCAACCCGCCACGCT
CCCCTTGCACATGGACTGGAAAGATGATAGCCT

Seq. 2:GACTACATCCCAGCAACAGTGACACATTCCACCGCGAGCCCAC
CCTCCACCTTCCCAACACTACTATTATTATTATTATTATTATTATTATTA TTATTATTATTATTATTATTATTATTATTTTTATTTTTTTACTTGGAGCA
ACCCGCCACGCTCCCCTTGCACATGGACTGGAAAGATGATAGCCT

By way of example, the (ATT)ₙ-sequence-contained marker can be identified by forward primer Seq. 3 and reverse primer Seq. 4 of primer pair 3a as follow:

Seq. 3: GACTACATCCCAGCAACAGTGA

Seq. 4: AGGCTATCATCTTTCCAGTCCA

By way of example, the cold hardiness marker can be selected from a (G)ₙ-sequence-contained marker as follows:

Seq. 5:ATGAGGTGAACAAAAGCCAATGGTTTCCAGACTCCACCCAAAC
ACAACCTTTTAGTGTTTTTTAAAATAAAGCCTAAATGGGGGGGGGGGGGG
TCGTGTTCAGCCCCCACTTGCTTACGCTTTACTAAGCGGCGAGTTAAGTC
TCTGTCAAGGA

Seq. 6:ATGAGGTGAACAAAAGCCAATGGTTTCCAGACTCCACCCAAAC
ACAACCTTTTAGTGTTTTTTAAAATAAAGCCTAAATGGGGGGGGGGGTCG
TGTTCAGCCCCCACTTGCTTACGCTTTACTAAGCGGCGAGTTAAGTCTCT
GTCAAGGA

By way of example, the (G)ₙ-sequence-contained marker can be identified by forward primer Seq. 7 and reverse primer Seq. 8 of primer pair 3a as follow:

Seq. 7: ATGAGGTGAACAAAAGCCAATGG

Seq. 8: TCCTTGACAGAGACTTAACTCGC

By way of example, the cold hardiness marker can be selected from a (GAG)ₙ-sequence-contained marker as follows:

Seq. 9:TATGGGCTGGAGAGGAGACTAAAGGAGGTGTGAGAGGAGGCGC
CTCCTTATAGGACAGTGCTCTCGCTCTCCAGGTCACAGTCTTTAGGAAGA
GGCGGCGAGGAGGAGGAGGAGGAGGAGAAGGCGGTGGCAGTCGGCAGCTG
CTGGTCTACTGCCGTGAAGTC

Seq. 10:TATGGGCTGGAGAGGAGACTAAAGGAGGTGTGAGAGGAGGCG
CCTCCTTATAGGACAGTGCTCTCGCTCTCCAGGTCACAGTCTTTAGGAAG
AGGCGGCGAGGAGGAGAAGGCGGTGGCAGTCGGCAGCTGCTGGTCTACTG
CCGTGAAGTC

By way of example, the (GAG)ₙ-sequence-contained marker can be identified by forward primer Seq. 11 and reverse primer Seq. 12 of primer pair 3a as follow:

Seq. 11: TATGGGCTGGAGAGGAGACTAA

Seq. 12: GACTTCACGGCAGTAGACCAG

By way of example, the cold hardiness marker can be selected from a (GT)ₙ-sequence-contained marker as follows:

Seq. 13:GAGCGTTGTTGTGTTTCTGCTATTAAGTGTGTGTGTGTGTGT
AGCACCAGCATGATGTCGGACTTTGACATCATGCTCGCCCGGAGGAAAGC
CATGAACAGCAAGAAGAG

Seq. 14:GAGCGTTGTTGTGTTTCTGCTATTAAAAAAGTGTGTGTGTGT GTGTGTGTGTGTGTGTGTGTGTGTGTGTAGCACTAGCGTGATGTCGGACT
TCGACATCATGCTCGCCCGGAGGAAAGCCATGAACAGCAAGAAGAG

By way of example, the (GT)ₙ-sequence-contained marker can be identified by forward primer Seq. 15 and reverse primer Seq. 16 of primer pair 3a as follow:

Seq. 15: GAGCGTTGTTGTGTTTCTGCTA

Seq. 16: CTCTTCTTGCTGTTCATGGCTT

By way of example, the cold hardiness marker can be selected from a (CA)ₙ-sequence-contained marker as follows:

Seq. 17:ACAGCAGGAGGAGAGGAGAGAGGGGAGGAGCAGACTGAACTT
CATAGACACCGGTCCCCTCGGCTCACCCCTCTCATTCACACACACACACA CACACACACACACAGTCGCACCTAATGTGAATACTTTGGGTTTGTGGCAG
CAGTGTGGATGTTTCTTTGTGTCGCTGAACT

Seq. 18:ACAGCAGGAGGAGAGGAGAGAGGGGAGGAGCAGACTGA
ACTTCATAGACACCGGTCCCCTCGGCTCACCCCTCTCATTCACACACACA CACACACACACACACACACACACACACACACACAGTCGCACCTAATGTGA
ATACTTTGGGTTTGTGGCAGCAGTGTGGATGTTTCTTTGTGTCGCTGAAC
T

By way of example, the (CA)ₙ-sequence-contained marker can be identified by forward primer Seq. 19 and reverse primer Seq. 20 of primer pair 3a as follow:

Seq. 19: ACAGCAGGAGGAGAGGAGAGA

Seq. 20: AGTTCAGCGACACAAAGAAACA

By way of example, the salinity tolerance marker can be selected from a (CA)₁₁-sequence-contained marker as follows:

Seq. 21:ACTCCCATACTGTATTTGCGTGTCTGTTTGTGTGTGCAGAGC
AGGTTCTCTGGTCATACACACGCACACACACACACACACACACACGCCCT
GTCGATTTCTTATTTCTGCTGATAAAGAAGAGGAGGCACTTGTGAAATGA
AC

By way of example, the (CA)₁₁-sequence-contained marker can be identified by forward primer Seq. 22 and reverse primer Seq. 23 of primer pair 3a as follow:

Seq. 22: ACTCCCATACTGTATTTGCGTGT

Seq. 23: GTTCATTTCACAAGTGCCTCCTC

By way of example, the salinity tolerance marker can be selected from a (AT)₈-sequence-contained marker as follows:

Seq. 24:AGCAGATTAACATTGAAATAGGCTGTAAAAAAAAAATA
GAGAAAAGCACAGAAAATAATAGACAAAGGTCTGTCTGAATATATATATA TATATATACATACACTATCTTAAAAATTAAAAAAAAAAACATTAAACATA
AGGTAGTGTAAAAACAAGTTGTATAGACAATGAAATACAAAATCATCTCA
TAAAAGAATTAATGTAATTCTGTGTTTCTGAATTTGCTTTTCTACTCACA
CAG

By way of example, the (AT)₈-sequence-contained marker can be identified by forward primer Seq. 25 and reverse primer Seq. 26 of primer pair 3a as follow:

Seq. 25: AGCAGATTAACATTGAAATAGGCT

Seq. 26: CTGTGTGAGTAGAAAAGCA

By way of example, the salinity tolerance marker can be selected from a (TAT)₈-sequence-contained marker as follows:

Seq. 27:TAAAGCAACTTTTGTGGGCAAGTTTGATCTGCTGGGTA TATTATTATTATTATTATTATTATTGTTATTGTTGGTTATTATACACATG
GTGGCTCTGCTTGTAAAGTGAAA

By way of example, the (TAT)₈-sequence-contained marker can be identified by forward primer Seq. 28 and reverse primer Seq. 29 of primer pair 3a as follow:

Seq. 28: TAAAGCAACTTTTGTGGGCAAGT

Seq. 29: TTTCACTTTACAAGCAGAGCCAC

By way of example, the salinity tolerance marker can be selected from a (TTTG)₅-sequence-contained marker as follows:

Seq. 30:AGGGTTTAAAGAGAGTTTGGCCTAGTCAACATATTTTT GTTTGTTTGTTTGTTTGTATGTATGTGTGTTTGTTTTGAATTGTGTTGAG
CTGAACTAGATTTTGCATCTCTATAAAGAATTCAGGAATCCACTGTCTAA
GGGCTA

By way of example, the (TTTG)₅-sequence-contained marker can be identified by forward primer Seq. 31 and reverse primer Seq. 32 of primer pair 3a as follow:

Seq. 31: AGGGTTTAAAGAGAGTTTGGCCT

Seq. 32: TAGCCCTTAGACAGTGGATTCCT

By way of example, the salinity tolerance marker can be selected from a (AT)₁₂-sequence-contained marker as follows:

Seq. 33:CAGTGTGAGTTTAACTTCGGGTCCAAGCCTTTCCGTCA
CCCATATATATATATATATATATATATGTATATATAATTTTTTTGTTATT
TTTTTGTGTATTTTTTATTTTTTTCCTCCCCAGTCTGGGATACCCACTAA
AATG

By way of example, the (AT)₁₂-sequence-contained marker can be identified by forward primer Seq. 34 and reverse primer Seq. 35 of primer pair 3a as follow:

Seq. 34: CAGTGTGAGTTTAACTTCG

Seq. 35: CATTTTAGTGGGTATCC

By way of example, the disease resistance marker can be selected from a (CAGG)₇-sequence-contained marker as follows:

Seq. 36:GCACAGACACAGTAACACATGCACTGAGTATTCATTTG
ACTGAGAGTATGTGCTAAATTTGATTTGGTTTTGTTTTCAGGCTTGTCAG
CTGCCATTTAAACATGCATAGACAGCCAGGCAGGCAGGCAGGCAGGCAGG CAGGCGTCTGTGTGTCCCTGTTTCTGTTTGAAGTGGATGATCTCTGATCC
CACGTACCTCTCTGATTGGGACATGCGTAGTCTCTCTCCGTCTGTCTGTT
TTCCTTCTTCACCCCACGAAAGCTTTCTGAATCGAGTCAGGCAAAGCTGA
AATAATACTGTTTGGAAAGGAAAATTAAATGACATAAAGAACTGTTTCTT
TAATAAATGAAATAGGAAGCATGTACCAGGGAGT

By way of example, the (CAGG)₇-sequence-contained marker can be identified by forward primer Seq. 37 and reverse primer Seq. 38 of primer pair 3a as follow:

Seq. 37: GCACAGACACAGTAACACATGCAC

Seq. 38: ACTCCCTGGTACATGCTTCCTA

By way of example, the disease resistance marker can be selected from a (CTAC)₇-sequence-contained marker as follows:

Seq. 39:CACCACTGTCAACTGGCTAATGGACCTAAGGGCAGACT ATCTATCTACCTATCTACCTACCTACCTACCTACCTACCTACGTACCTAC CTACAAACAATACACATGCACCCCAACTAATATTTGTTTAAGTGTCCCTT
GGGAATCTGCACCCCAACCACATGCTTTGGTAGCCTGTCTAAACTGTTTT
AACTTGCAACATATATCAGGGTTTGATACCTGCCAACAGTAGTGAAATCT
CATATTGAATTAAATGACCAAAATCTTCTTCAAAGTAGCTTTATACTCGA
ATCGATTTTCTAGAATTAAATTCAAACATATTTTACAGTTGTGATAGTGC
CTGCGGTATCAAGAAGGTAAC

By way of example, the (CTAC)₇-sequence-contained marker can be identified by forward primer Seq. 40 and reverse primer Seq. 41 of primer pair 3a as follow:

Seq. 40: CACCACTGTCAACTGGCTAATG

Seq. 41: GTTACCTTCTTGATACCGCAGG

By way of example, the disease resistance marker can be selected from a (TTGA)₂₀-sequence-contained marker as follows:

Seq. 42:GGACCCTGAATCTTCCCTTAGTTATGCTGCAAAAAGTG
TAGAATGCTGGGAGATTCCCATGATGCATTGTGTATTTCTTCTTCAGTCG
TCTTTTTGATTGATTGATTGATTGATTGATTGATTGATTGATTGATTGAT TGATTGATTGATTGATTGATTGATTGATTGATTGACTGGAGGAGAGCAGT
TTGAGAACAGGAAAAAACAGCCATCTGGTCCTGCAGCCATACTCCGTCGC
TGGAATCAGACCCACCTGGCTGCATCGAGTGGAAGTAGTCATTTATATTG
TATATTGTATATTTCTGTCTGAGTACATGTGCATGTGTGCGTAAGTGTCT
GTATCTTGTGTCCAACTCAAGAGAGAGAGCC

By way of example, the (TTGA)₂₀-sequence-contained marker can be identified by forward primer Seq. 43 and reverse primer Seq. 44 of primer pair 3a as follow:

Seq. 43: GGACCCTGAATCTTCCCTTAGT

Seq. 44: GGCTCTCTCTCTTGAGTTGGACA

By way of example, the disease resistance marker can be selected from a (TTGT)₁₀-sequence-contained marker as follows:

Seq. 45:ACTAGCAGATGATAAATGCGCCAGAAAATAAACGCTTA
AAGGGAATATGCACCAGTCCAGCTGTTTGTTTGTTTGTTTGTTTGTTTGT TTGTTTGTTTGTTTTACTGCTCAAATTTTGTATTTGCACTGTTTCGGCTC
AGTGTGACAGTTTTTTTGGTGTCAGCTCAGATTGCGAGGACATCTCTTGT
GCGCAGACCTCTTCAGGGCACCCCACAGATTTTCTGTAGGATTTAGGTCT
GGGCAT

By way of example, the (TTGT)₁₀-sequence-contained marker can be identified by forward primer Seq. 46 and reverse primer Seq. 47 of primer pair 3a as follow:

Seq. 46: ACTAGCAGATGATAAATGCGC

Seq. 47: ATGCCCAGACCTAAATCCTACA

By way of example, the disease resistance marker can be selected from a (GAAAA)₆-sequence-contained marker as follows:

Seq. 48:TGTATGTAGCCGAAGTAGCCAATCTCAACAGTACTTGC
TTGTTTTAATTTATTTACTGGTTTGGTGCACTTTGTATTTAAGAAAAAAA
ATTGATAACTGTAAACCACCATGGCAGACCCGATGGCATTTTCATGCTTC
GCAACATCATTAGCGAATGATGTCTGTTCTGAAAAGAAAAGAAAAGAAAA GAAAAGAAAAGACCTTGACGTCCGTGGAAGTCAACAGCGGTGGATGACTG
CAGAATCATTCGTGCGATCATCCACCACTGTTGAGAAACCCCTTCACAAC
AGCCAACAAAATGAACAACACTCTGCAGGAGGAAGGCGTATCCAAGTATA
CCATAAAGAGAAGACTGCAGGTAAGTAAATACAAAGGGTTCACTGCAAGG

By way of example, the (GAAAA)₆-sequence-contained marker can be identified by forward primer Seq. 49 and reverse primer Seq. 50 of primer pair 3a as follow:

Seq. 49: TGTATGTAGCCGAAGTAGCCAA

Seq. 50: CCTTGCAGTGAACCCTTTGTAT

Referring back to FIGS. 1, 2 and 3, by way of example, the biomolecular characteristic identification method in accordance with the preferred embodiment of the present invention includes: automatically, semi-automatically or manually transmitting the identification result 40 to a device (e.g., computer display device, mobile communication device or other devices) for displaying a correct source of original aquatic strain or product or an incorrect source of original aquatic strain or product.

FIG. 4 is a schematic block diagram of an exogenous biomolecular tracing system applied in the biomolecular characteristic identification method and system thereof for Taiwan Tilapia strains in accordance with a preferred embodiment of the present invention. Turning now to FIG. 4, an exogenous biomolecular tracing system in accordance with the preferred embodiment of the present invention includes a plurality of feature sequence markers 11, a basic material 12, a plurality of Taiwan Tilapia organisms (exogenous aquatic creatures or products) 2 and at least one primer pair 30.

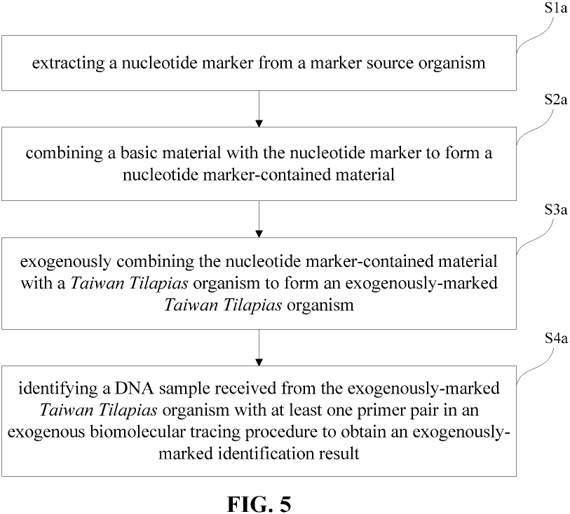

FIG. 5 is a flow chart of an exogenous biomolecular tracing system applied in the biomolecular characteristic identification method and system thereof for Taiwan Tilapia strains in accordance with a preferred embodiment of the present invention. Turning now to FIGS. 4 and 5, the exogenous biomolecular tracing method in accordance with the preferred embodiment of the present invention includes the step S1a: automatically, semi-automatically or manually extracting the feature sequence marker 11 from a marker source organism 10 with the extraction device 1.

With continued reference to FIGS. 4 and 5, by way of example, the feature sequence marker (nucleotide marker) 11 can be selected from a DNA extract, a DNA extract liquid, a DNA extract powder (e.g., freeze-dried powder), a DNA extract-related material or combinations thereof.

Still referring to FIGS. 4 and 5, by way of example, the exogenous biomolecular tracing method in accordance with the preferred embodiment of the present invention includes the step S2a: automatically, semi-automatically or manually combining the basic material 12 with the feature sequence marker 11 in an auto-device (i.e., feeder device, stirrer device or combination thereof) to form a nucleotide marker-contained material.

Still referring to FIGS. 4 and 5, by way of example, the exogenous biomolecular tracing method in accordance with the preferred embodiment of the present invention includes the step S3a: exogenously combining the nucleotide marker-contained material with the Taiwan Tilapia organisms 2 in an auto-device (i.e., feed-supplying device, dipping device, painting device, sprayer device or combinations thereof) to form an exogenously-marked Taiwan Tilapia organism.

Still referring to FIGS. 4 and 5, by way of example, exogenously combining the nucleotide marker-contained material with the Taiwan Tilapia organisms 2 includes a feeding process, a dipping process, a painting process, a spraying process or combinations thereof. In another embodiment, by way of example, the nucleotide marker-contained material can be selectively formed as an oral vaccine or a vaccine product.

Still referring to FIGS. 4 and 5, by way of example, the exogenous biomolecular tracing method in accordance with the preferred embodiment of the present invention includes the step S4a: automatically, semi-automatically or manually identifying a DNA sample received from the exogenously-marked Taiwan Tilapia organism (e.g., aquatic creature or aquatic product) with at least one primer pair 30 in an exogenous biomolecular tracing procedure with the DNA testing device 3 to obtain an exogenously-marked identification result 4.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A biomolecular characteristic identification system for Taiwan Tilapia strains comprising:
a predetermined environment conditioned and provided to breed and train a plurality of Taiwan Tilapias to obtain a plurality of baited Taiwan Tilapias;
at least one feature sequence marker obtained from seeking at least one nucleotide mark in the baited Taiwan Tilapias;
at least one designed primer pair corresponding to the feature sequence marker for identification and produced according to the feature sequence marker; and
an identification result obtained from identifying an unknown DNA sample of the baited Taiwan Tilapias with the at least one primer pair in a biomolecular tracing procedure;
wherein the identification result shows a correct source of original baited Taiwan Tilapias or an incorrect source of original baited Taiwan Tilapias.

2. The biomolecular characteristic identification system as defined in claim 1, wherein the feature sequence marker is selected from a cold hardiness marker, a disease resistance marker, a salinity tolerance marker or combinations thereof.

3. The biomolecular characteristic identification system as defined in claim 2, wherein the cold hardiness marker is selected from a (ATT)ₙ-sequence-contained marker, a (G)ₙ-sequence-contained marker, a (GAG)ₙ-sequence-contained marker, a (GT)ₙ-sequence-contained marker or a (CA)ₙ-sequence-contained marker.

4. The biomolecular characteristic identification system as defined in claim 2, wherein the salinity tolerance marker is selected from a (CA)₁₁-sequence-contained marker, a (AT)₈-sequence-contained marker, a (TAT)₈-sequence-contained marker, a (TTTG)₅-sequence-contained marker or a (AT)₁₂-sequence-contained marker.

5. The biomolecular characteristic identification system as defined in claim 2, wherein the disease resistance marker is selected from a (CAGG)₇-sequence-contained marker, a (CTAC)₇-sequence-contained marker, a (TTGA)₂₀-sequence-contained marker, a (TTGT)₁₀-sequence-contained marker or a (GAAAA)₆-sequence-contained marker.

6. A biomolecular characteristic identification method for Taiwan Tilapia strains comprising:
breeding and training a plurality of Taiwan Tilapias in a predetermined environment to obtain a plurality of baited Taiwan Tilapias;
seeking at least one nucleotide mark in the baited Taiwan Tilapias to obtain at least one feature sequence marker therefrom;
producing at least one design of primer pair according to the feature sequence marker for identification; and
identifying an unknown DNA sample of the baited Taiwan Tilapias with the at least one primer pair in a biomolecular tracing procedure to obtain an identification result.

7. The biomolecular characteristic identification method as defined in claim 6, wherein the feature sequence marker is selected from a cold hardiness marker, a disease resistance marker, a salinity tolerance marker or combinations thereof.

8. The biomolecular characteristic identification method as defined in claim 7, wherein the cold hardiness marker is selected from a (ATT)ₙ-sequence-contained marker, a (G)ₙ-sequence-contained marker, a (GAG)ₙ-sequence-contained marker, a (GT)ₙ-sequence-contained marker or a (CA)ₙ-sequence-contained marker.

9. The biomolecular characteristic identification method as defined in claim 7, wherein the salinity tolerance marker is selected from a (CA)ₙ-sequence-contained marker, a (AT)₈-sequence-contained marker, a (TAT)₈-sequence-contained marker, a (TTTG)₅-sequence-contained marker or a (AT)₁₂-sequence-contained marker.

10. The biomolecular characteristic identification method as defined in claim 7, wherein the disease resistance marker is selected from a (CAGG)₇-sequence-contained marker, a (CTAC)₇-sequence-contained marker, a (TTGA)₂₀-sequence-contained marker, a (TTGT)₁₀-sequence-contained marker or a (GAAAA)₆-sequence-contained marker.

11. The biomolecular characteristic identification method as defined in claim 6, further comprising an exogenous biomolecular tracing method comprising:
extracting a first nucleotide marker from a first marker source organism;
combining a first basic material with the first nucleotide marker to form a first nucleotide marker-contained material;
extracting a second nucleotide marker from a second marker source organism;
combining a second basic material with the second nucleotide marker to form a second nucleotide marker-contained material;
in a first stage, exogenously combining the first nucleotide marker-contained material with an aquatic creature or an aquatic product to form a first exogenously-marked aquatic creature or a first exogenously-marked aquatic product;
in a second stage, exogenously combining the second nucleotide marker-contained material with the first exogenously-marked aquatic creature or the first exogenously-marked aquatic product to form a second exogenously-marked aquatic creature or a second exogenously-marked aquatic product; and
identifying a DNA sample received from the second exogenously-marked aquatic creature or the second exogenously-marked aquatic product with at least one primer pair in an exogenous biomolecular tracing procedure to obtain an exogenously-marked identification result.

12. The method as defined in claim 11, wherein the first or second nucleotide marker is selected from a DNA extract, a DNA extract liquid, a DNA extract powder, a DNA extract-related material or combinations thereof.

13. The method as defined in claim 11, wherein exogenously combining the first or second nucleotide marker-contained material with the aquatic creature or the aquatic product includes a feeding process, a dipping process, a painting process, a spraying process or combinations thereof.

14. The method as defined in claim 11, wherein the at least one primer pair is applied to execute a test of polymerase chain reaction (PCR) or loop-mediated isothermal amplification (LAMP).

15. The method as defined in claim 11, wherein the exogenously-marked identification result includes a combination set of codes or code forms for tracing the second exogenously-marked aquatic creature or the second exogenously-marked aquatic product.


SEQUENCE LISTING (50 sequences)

SEQ ID NO: 1 - DNA, 176 bp, Unknown
  Taiwan Tilapia sequence (cold hardiness marker 1-1)
gactacatcccagcaacagtgacacattccaccgcgagcccaccctccaccttcccaacactaccattattattattattattattattattattattattattattatttgtatttttttacttggagcaacccgccacgctccccttgcacatggactggaaagatgatagcct

SEQ ID NO: 2 - DNA, 188 bp, Unknown
  Taiwan Tilapia sequence (cold hardiness marker 1-2)
gactacatcccagcaacagtgacacattccaccgcgagcccaccctccaccttcccaacactactattattattattattattattattattattattattattattattattattattatttttatttttttacttggagcaacccgccacgctccccttgcacatggactggaaagatgatagcct

SEQ ID NO: 3 - DNA, 22 bp, artificial sequence
  Forward primer
gactacatcccagcaacagtga

SEQ ID NO: 4 - DNA, 22 bp, artificial sequence
  Reverse primer
aggctatcatctttccagtcca

SEQ ID NO: 5 - DNA, 154 bp, Unknown
  Taiwan Tilapia sequence (cold hardiness marker 2-1)
atgaggtgaacaaaagccaatggtttccagactccacccaaacacaaccttttagtgttttttaaaataaagcctaaatggggggggggggggtcgtgttcagcccccacttgcttacgctttactaagcggcgagttaagtctctgtcaagga

SEQ ID NO: 6 - DNA, 151 bp, Unknown
  Taiwan Tilapia sequence (cold hardiness marker 2-2)
atgaggtgaacaaaagccaatggtttccagactccacccaaacacaaccttttagtgttttttaaaataaagcctaaatgggggggggggtcgtgttcagcccccacttgcttacgctttactaagcggcgagttaagtctctgtcaagga

SEQ ID NO: 7 - DNA, 23 bp, artificial sequence
  Forward primer
atgaggtgaacaaaagccaatgg

SEQ ID NO: 8 - DNA, 23 bp, artificial sequence
  Reverse primer
tccttgacagagacttaactcgc

SEQ ID NO: 9 - DNA, 164 bp, Unknown
  Taiwan Tilapia sequence (cold hardiness marker 3-1)
tatgggctggagaggagactaaaggaggtgtgagaggaggcgcctccttataggacagtgctctcgctctccaggtcacagtctttaggaagaggcggcgaggaggaggaggaggaggagaaggcggtggcagtcggcagctgctggtctactgccgtgaagtc

SEQ ID NO: 10 - DNA, 152 bp, Unknown
  Taiwan Tilapia sequence (cold hardiness marker 3-2)
tatgggctggagaggagactaaaggaggtgtgagaggaggcgcctccttataggacagtgctctcgctctccaggtcacagtctttaggaagaggcggcgaggaggagaaggcggtggcagtcggcagctgctggtctactgccgtgaagtc

SEQ ID NO: 11 - DNA, 22 bp, artificial sequence
  Forward primer
tatgggctggagaggagactaa

SEQ ID NO: 12 - DNA, 21 bp, artificial sequence
  Reverse primer
gacttcacggcagtagaccag

SEQ ID NO: 13 - DNA, 110 bp, Unknown
  Taiwan Tilapia sequence (cold hardiness marker 4-1)
gagcgttgttgtgtttctgctattaagtgtgtgtgtgtgtgtagcaccagcatgatgtcggactttgacatcatgctcgcccggaggaaagccatgaacagcaagaagag

SEQ ID NO: 14 - DNA, 138 bp, Unknown
  Taiwan Tilapia sequence (cold hardiness marker 4-2)
gagcgttgttgtgtttctgctattaaaaaagtgtgtgtgtgtgtgtgtgtgtgtgtgtgtgtgtgtgtgtagcactagcgtgatgtcggacttcgacatcatgctcgcccggaggaaagccatgaacagcaagaagag

SEQ ID NO: 15 - DNA, 22 bp, artificial sequence
  Forward primer
gagcgttgttgtgtttctgcta

SEQ ID NO: 16 - DNA, 22 bp, artificial sequence
  Reverse primer
ctcttcttgctgttcatggctt

SEQ ID NO: 17 - DNA, 173 bp, Unknown
  Taiwan Tilapia sequence (cold hardiness marker 5-1)
acagcaggaggagaggagagaggggaggagcagactgaacttcatagacaccggtcccctcggctcacccctctcattcacacacacacacacacacacacacacagtcgcacctaatgtgaatactttgggtttgtggcagcagtgtggatgtttctttgtgtcgctgaact

SEQ ID NO: 18 - DNA, 189 bp, Unknown
  Taiwan Tilapia sequence (cold hardiness marker 5-2)
acagcaggaggagaggagagaggggaggagcagactgaacttcatagacaccggtcccctcggctcacccctctcattcacacacacacacacacacacacacacacacacacacacacacagtcgcacctaatgtgaatactttgggtttgtggcagcagtgtggatgtttctttgtgtcgctgaact

SEQ ID NO: 19 - DNA, 21 bp, artificial sequence
  Forward primer
acagcaggaggagaggagaga

SEQ ID NO: 20 - DNA, 22 bp, artificial sequence
  Reverse primer
agttcagcgacacaaagaaaca

SEQ ID NO: 21 - DNA, 144 bp, Unknown
  Taiwan Tilapia sequence (salinity tolerance marker 1)
actcccatactgtatttgcgtgtctgtttgtgtgtgcagagcaggttctctggtcatacacacgcacacacacacacacacacacacgccctgtcgatttcttatttctgctgataaagaagaggaggcacttgtgaaatgaac

SEQ ID NO: 22 - DNA, 23 bp, artificial sequence
  Forward primer
actcccatactgtatttgcgtgt

SEQ ID NO: 23 - DNA, 23 bp, artificial sequence
  Reverse primere
gttcatttcacaagtgcctcctc

SEQ ID NO: 24 - DNA, 241 bp, Unknown
  Taiwan Tilapia sequence (salinity tolerance marker 2)
agcagattaacattgaaataggctgtaaaaaaaaaatagagaaaagcacagaaaataatagacaaaggtctgtctgaatatatatatatatatatacatacactatcttaaaaattaaaaaaaaaaacattaaacataaggtagtgtaaaaacaagttgtatagacaatgaaatacaaaatcatctcataaaagaattaatgtaattctgtgtttctgaatttgcttttctactcacacag

SEQ ID NO: 25 - DNA, 24 bp, artificial sequence
  Forward primer
agcagattaacattgaaataggct

SEQ ID NO: 26 - DNA, 19 bp, artificial sequence
  Reverse primer
ctgtgtgagtagaaaagca

SEQ ID NO: 27 - DNA, 111 bp, Unknown
  Taiwan Tilapia sequence (salinity tolerance marker 3)
taaagcaacttttgtgggcaagtttgatctgctgggtatattattattattattattattattgttattgttggttattatacacatggtggctctgcttgtaaagtgaaa

SEQ ID NO: 28 - DNA, 23 bp, artificial sequence
  Forward primer
taaagcaacttttgtgggcaagt

SEQ ID NO: 29 - DNA, 23 bp, artificial sequence
  Reverse primer
tttcactttacaagcagagccac

SEQ ID NO: 30 - DNA, 144 bp, Unknown
  Taiwan Tilapia sequence (salinity tolerance marker 4)
agggtttaaagagagtttggcctagtcaacatatttttgtttgtttgtttgtttgtatgtatgtgtgtttgttttgaattgtgttgagctgaactagattttgcatctctataaagaattcaggaatccactgtctaagggcta

SEQ ID NO: 31 - DNA, 23 bp, artificial sequence
  Forward primer
agggtttaaagagagtttggcct

SEQ ID NO: 32 - DNA, 23 bp, artificial sequence
  Reverse primer
tagcccttagacagtggattcct

SEQ ID NO: 33 - DNA, 142 bp, Unknown
  Taiwan Tilapia sequence (salinity tolerance marker 1)
cagtgtgagtttaacttcgggtccaagcctttccgtcacccatatatatatatatatatatatatgtatatataatttttttgttatttttttgtgtattttttatttttttcctccccagtctgggatacccactaaaatg

SEQ ID NO: 34 - DNA, 19 bp, artificial sequence
  Forward primer
cagtgtgagtttaacttcg

SEQ ID NO: 35 - DNA, 17 bp, artificial sequence
  Reverse primer
cattttagtgggtatcc

SEQ ID NO: 36 - DNA, 372 bp, Unknown
  Taiwan Tilapia sequence (disease resistance marker 1)
gcacagacacagtaacacatgcactgagtattcatttgactgagagtatgtgctaaatttgatttggttttgttttcaggcttgtcagctgccatttaaacatgcatagacagccaggcaggcaggcaggcaggcaggcaggcgtctgtgtgtccctgtttctgtttgaagtggatgatctctgatcccacgtacctctctgattgggacatgcgtagtctctctccgtctgtctgttttccttcttcaccccacgaaagctttctgaatcgagtcaggcaaagctgaaataatactgtttggaaaggaaaattaaatgacataaagaactgtttctttaataaatgaaataggaagcatgtaccagggagt

SEQ ID NO: 37 - DNA, 24 bp, artificial sequence
  Forward primer
gcacagacacagtaacacatgcac

SEQ ID NO: 38 - DNA, 22 bp, artificial sequence
  Reverse primer
actccctggtacatgcttccta

SEQ ID NO: 39 - DNA, 359 bp, Unknown
  Taiwan Tilapia sequence (disease resistance marker 2)
caccactgtcaactggctaatggacctaagggcagactatctatctacctatctacctacctacctacctacctacctacgtacctacctacaaacaatacacatgcaccccaactaatatttgtttaagtgtcccttgggaatctgcaccccaaccacatgctttggtagcctgtctaaactgttttaacttgcaacatatatcagggtttgatacctgccaacagtagtgaaatctcatattgaattaaatgaccaaaatcttcttcaaagtagctttatactcgaatcgattttctagaattaaattcaaacatattttacagttgtgatagtgcctgcggtatcaagaaggtaac

SEQ ID NO: 40 - DNA, 22 bp, artificial sequence
  Forward primer
caccactgtcaactggctaatg

SEQ ID NO: 41 - DNA, 22 bp, artificial sequence
  Reverse primer
gttaccttcttgataccgcagg

SEQ ID NO: 42 - DNA, 369 bp, Unknown
  Taiwan Tilapia sequence (disease resistance marker 3)
ggaccctgaatcttcccttagttatgctgcaaaaagtgtagaatgctgggagattcccatgatgcattgtgtatttcttcttcagtcgtctttttgattgattgattgattgattgattgattgattgattgattgattgattgattgattgattgattgattgattgattgactggaggagagcagtttgagaacaggaaaaaacagccatctggtcctgcagccatactccgtcgctggaatcagacccacctggctgcatcgagtggaagtagtcatttatattgtatattgtatatttctgtctgagtacatgtgcatgtgtgcgtaagtgtctgtatcttgtgtccaactcaagagagagagcc

SEQ ID NO: 43 - DNA, 22 bp, artificial sequence
  Forward primer
ggaccctgaatcttcccttagt

SEQ ID NO: 44 - DNA, 23 bp, artificial sequence
  Reverse primer
ggctctctctcttgagttggaca

SEQ ID NO: 45 - DNA, 244 bp, Unknown
  Taiwan Tilapia sequence (disease resistance marker 4)
actagcagatgataaatgcgccagaaaataaacgcttaaagggaatatgcaccagtccagctgtttgtttgtttgtttgtttgtttgtttgtttgtttgttttactgctcaaattttgtatttgcactgtttcggctcagtgtgacagtttttttggtgtcagctcagattgcgaggacatctcttgtgcgcagacctcttcagggcaccccacagattttctgtaggatttaggtctgggcat

SEQ ID NO: 46 - DNA, 21 bp, artificial sequence
  Forward primer
actagcagatgataaatgcgc

SEQ ID NO: 47 - DNA, 22 bp, artificial sequence
  Reverse primer
atgcccagacctaaatcctaca

SEQ ID NO: 48 - DNA, 388 bp, Unknown
  Taiwan Tilapia sequence (disease resistance marker 5)
tgtatgtagccgaagtagccaatctcaacagtacttgcttgttttaatttatttactggtttggtgcactttgtatttaagaaaaaaaattgataactgtaaaccaccatggcagacccgatggcattttcatgcttcgcaacatcattagcgaatgatgtctgttctgaaaagaaaagaaaagaaaagaaaagaaaagaccttgacgtccgtggaagtcaacagcggtggatgactgcagaatcattcgtgcgatcatccaccactgttgagaaaccccttcacaacagccaacaaaatgaacaacactctgcaggaggaaggcgtatccaagtataccataaagagaagactgcaggtaagtaaatacaaagggttcactgcaagg

SEQ ID NO: 49 - DNA, 22 bp, artificial sequence
  Forward primer
tgtatgtagccgaagtagccaa

SEQ ID NO: 50 - DNA, 22 bp, artificial sequence
  Reverse primer
ccttgcagtgaaccctttgtat